(19) United States
(12) Patent Application Publication
(10) Pub. No.: US 2009/0081772 A1
(43) Pub. Date: Mar. 26, 2009

(54) Method and device for collecting cellular material from cells isolated on a filter

(75) Inventor: Yvon Cayre, Paris (FR)

Correspondence Address: YOUNG & THOMPSON, 209 Madison Street, Suite 500, ALEXANDRIA, VA, 22314, US

(73) Assignee: METAGENEX, PARIS (FR)

(21) Appl. No.: 12/016,222

(22) Filed: Jan. 18, 2008

(30) Foreign Application Priority Data
Sep. 21, 2007 (FR) 07/57779

(51) Int. Cl. C12M 3/00; C07H 21/04

(52) U.S. Cl. 435/306.1; 536/25.41

(57) ABSTRACT

Method for collecting cellular material of particular cells present in a liquid includes:

a step (210) of introducing the liquid into a compartment via an upper opening of the compartment said compartment having a lower opening, a filter being positioned between the two openings which has micropores of a diameter intermediate between that of said particular cells and that of other cells,

a filtering step (215) during which most of the liquid and most of the other cells pass through the filter,

a step (230) of lysing the cells retained or: the filter and

a step (245, 230) of collecting cellular material from the lysed cells, on the filter.

The present invention concerns a method and a device for collecting cellular material from cells isolated on a filter. It applies, in particular, to collecting the genetic material from particular cells present in a liquid, in particular blood.

Certain particular blood cells, for example tumor or fetal cells, are in very low concentration and must be concentrated for cytopathalogical analysis. However, they are of larger size than blood cells.

It is known, for example from the document PCT/FR 2006/000562, that a formaldehyde-based fixation buffer can be applied to a blood sample in order to fix the cells searched for, then to pass the resulting liquid through a porous filter. This filter is then analyzed in a laboratory to search therein for the cells under a microscope. It is possible, subsequently, to extract them on the filter for anlyses, for example by a genetic analysis. However, this procedure cannot be used on a large scale and at a reasonable cost, due to the time, materials and precision of work that it involves.

This would allow molecular analyses to be conducted both on tumor cells and trophoblastic cells.

The present invention aims to mitigate these drawbacks and to meet this need by enabling collection, under conditions compatible with routine laboratory examinations, of a large proportion of the cellular material, in particular, RNA and DNA, of the cells considered, in good state.

To that end, according to a first aspect, the present invention concerns a method for collecting cellular material of particular cells present in a liquid, characterized in that it comprises:

a step of introducing the liquid into a compartment via an upper opening of the compartment, said compartment having a lower opening, a filter being positioned between the two openings which has micropores of a diameter intermediate between that of said particular cells and that of other cells,

a filtering step during which most of the liquid and of said other cells passes through the filter,

a step of lysing the cells retained on the filter and

a step of collecting cellular material from the lysed cells, on the filter.

In general, the cellular material collected by virtue of the implementation of the present invention is the genetic material of the cells. The method of the present invention thus enables the collection, directly on the filter and practically without loss, of the genetic material of rare cells, down to a single isolated cell. A high proportion of the genetic material of the cells considered is thus collected, in good state, under conditions compatible with routine laboratory examinations.

According to particular features, the method as succinctly set forth above comprises, prior to said filtering step, a step of applying a fixation buffer without formaldehyde to the liquid containing the particular cells.

Thus, the particular cells searched for are specifically hardened, without altering the genetic material.

According to particular features, during the introducing step, the compartment has the general form of a syringe in which the filter is positioned between the two openings.

By virtue of these provisions, a plunger may be applied in the upper opening without modifying the position of the filter.

According to particular features, during the filtration step, suction is applied by pressure reduction below the filter. By virtue of these provisions, the filtration is more effective and faster than without suction.

According to particular features, following the filtering step and prior to the collecting step, the compartment is placed on an Eppendorf type tube. A direct passage is thus established from the filter to a tube of Eppendorf type.

According to particular features, prior to the collecting step, a plunger is positioned in the upper opening of the compartment, comprising a central awl moveable within the plunger.

According to particular features, the moveable central awl has a pointed lower end.

According to particular features, the pointed lower end is star-shaped.

According to particular features, during the collecting step, the upper part of the awl is pressed to perforate the filter with the point of the awl.

According to particular features, the piston comprising a means for longitudinal blocking of the awl, during the collecting step, the awl is made to pivot within the plunger to free said blocking means, before pressing on the upper part thereof.

According to particular features, during the collecting step, the plunger is pressed vertically downwards to pass the content of the compartment into a tube placed below the compartment.

By virtue of each of these provisions, the cellular material of the particular cells may be extracted without deterioration via the filter perforation.

According to particular features, following the filtering step and prior to the collecting step, the content of the compartment is isolated by plugging the lower opening with a membrane.

According to particular features, said membrane is positioned below a bar surrounding the lower opening of each compartment. Fluid-tightness and keeping the lysis liquid above the filter are thus ensured.

According to particular features, said membrane is an adhesive membrane.

By virtue of each of these provisions, the content of the compartment is kept away from the environment between the filtration and the collection of the cellular material of the particular cells.

According to particular features, during the collecting step, the upper part of the awl is pressed to perforate the membrane isolating the content of the compartment.

According to particular features, the filter is made of polycarbonate with a hydrophilic surface treatment. The use of such a filter improves the degree that the particular cells are retained and reduces the adherence of the cellular material to collect.

According to particular features, the filter has a baseline pore diameter of 7.5 μm. Thus, due to the dispersion in the diameters, practically no pore has a diameter greater than 8 μm.

The use of this pore diameter, which is less than the pore diameter traditionally used for cytological analysis filters, makes it possible to space the pores apart, which reduces the number of coalescent pores and avoids loss of particular cells.

According to a second aspect, the present invention concerns a device for collecting genetic material of particular cells present in a liquid, characterized in that it comprises:

a compartment comprising an upper opening and a lower opening, a filter being positioned between the two openings which has micropores of a diameter intermediate between that of said particular cells and that of other cells,

a means for introducing the liquid into said compartment via the upper opening,

a filtering means for passing most of the liquid and of said other cells through the filter,

a means for lysing the cells retained on the filter and

a means for collecting cellular material from the lysed cells, on the filter.

As the advantages, objects and features of this device are similar to those of the method of the present invention, as succinctly set forth above, they are not reviewed here.

Other advantages, objects and features of the present invention will emerge from the following description, given, with an explanatory purpose that is in no way limiting, with regard to the accompanying drawings, in which:

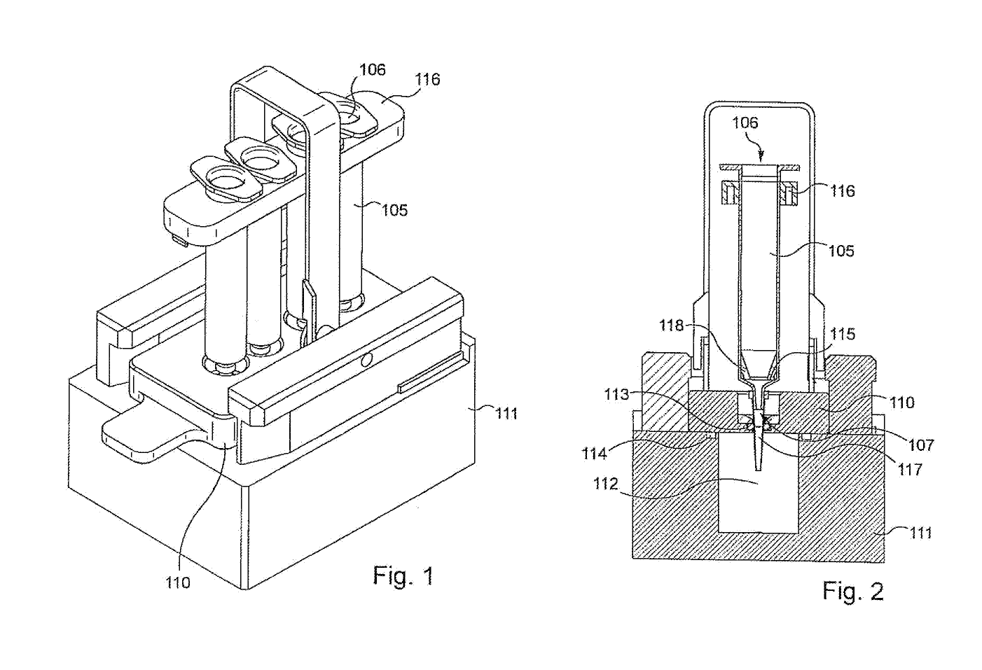

FIG. 1 is a diagram in perspective of parts of two particular embodiments of the device of the present invention, implemented in a first phase of the method of the present invention,

FIG. 2 is a diagram in cross-section of parts of the two specific embodiments of the device of the present invention, implemented in a first phase of the method of the present invention,

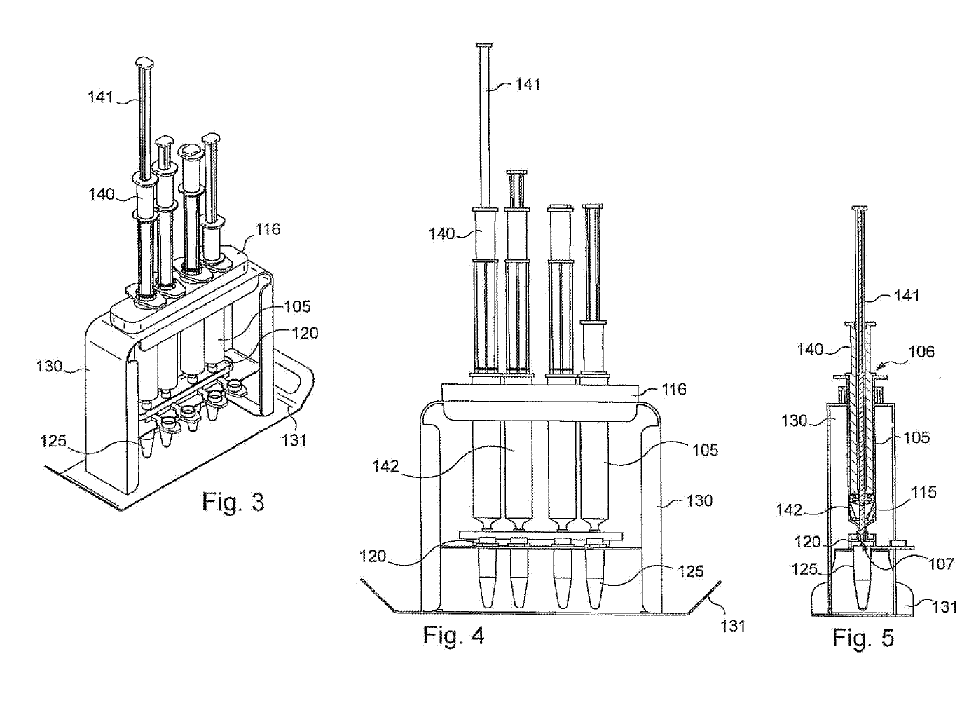

FIG. 3 is a diagram in perspective of parts of a first specific embodiment of the device of the present invention,

FIG. 4 is a diagram in elevation of parts of the first specific embodiment of the device of the present invention,

FIG. 5 is a diagram in cross-section of parts of the first particular embodiment of the device of the present invention,

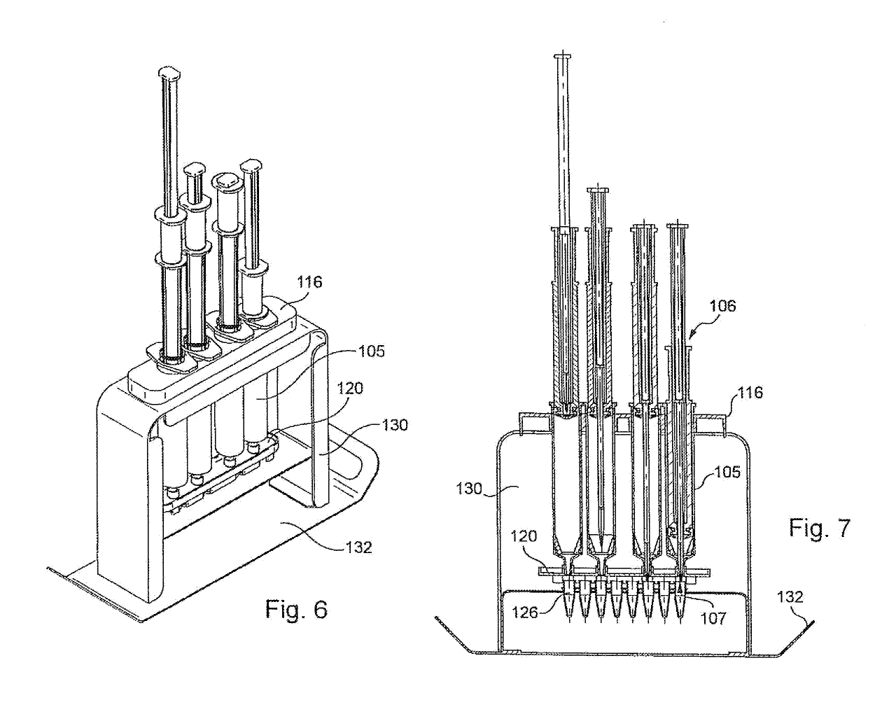

FIG. 6 is a diagram in perspective of parts of a second specific embodiment of the device of the present invention,

FIG. 7 is a diagram in cross-section of parts of the second specific embodiment of the device of the present invention,

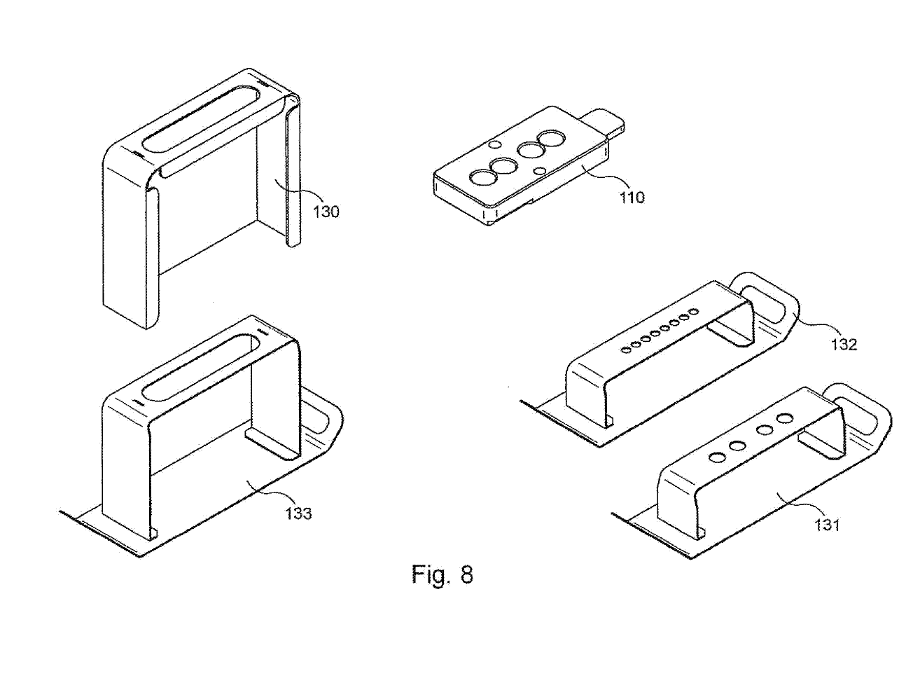

FIG. 8 is a diagram in perspective of parts of the two particular specific embodiments of the device of the present invention, used in a second phase of the method of the present invention, and

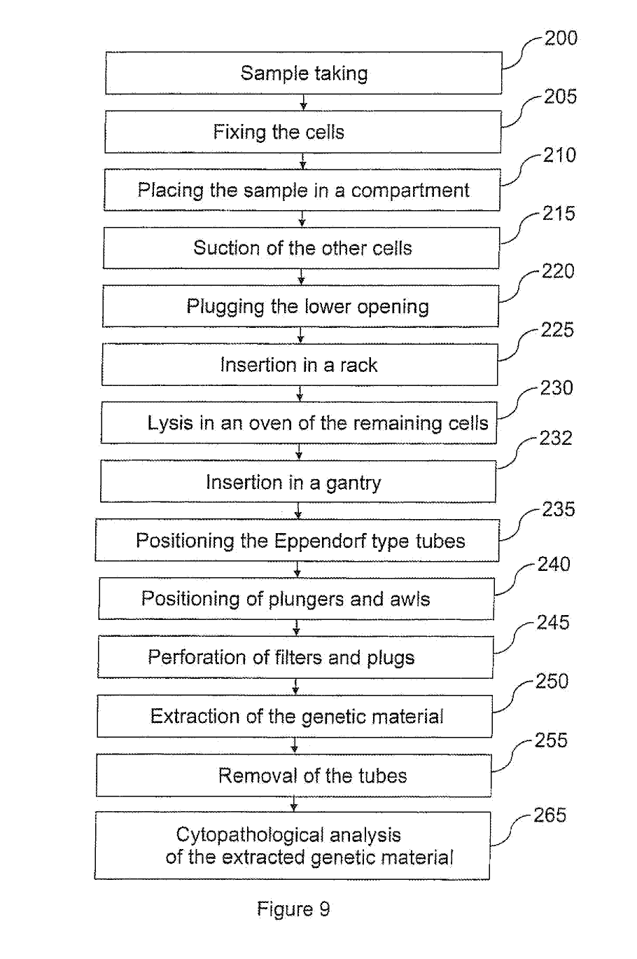

FIG. 9 represents, in logigram form, steps implemented in a particular embodiment of the method of the present invention.

As can be seen with regard to FIGS. 1 and 2, in the two embodiments illustrated in the drawings, the device for collecting cellular material, here genetic, comprises compartments (here four) of syringe form 105 each having an upper opening 106 and a lower opening 107. A washer or ring 118 placed in the lower opening 107 retains a filter 115. The filters 115 are micro-perforated and bonded onto the washers or rings 118 then inserted at the distal end, that is to say at the bottom, of the four compartments of syringe form 105. For example, the filter 115 made of polycarbonate with a hydrophilic surface treatment. The use of such a filter improves the level of retention of the particular cells and reduces the adherence of the cellular material to collect. The filter 115 has, for example, a 7.5 μm baseline pore diameter. Due to the dispersion in the diameters, practically no pore thus has a diameter greater than 8 μm.

At the small opening, or lower opening, of each compartment 105 a tip 117 is placed, in fluid-tight manner, in order to avoid potential contamination of the end of the compartment 105 by splashes coming from the reservoir 112. These compartments 105 are assembled by a bar 116, of plastics material, to form a single piece. The assembly thus constituted by the four compartments 105, by the upper bar 116, the four plungers 140 and the bar-plug 120 (described later) is for single use.

In the first phase of the method for collecting cellular material, the compartments 105 are supported by a plate 110 inserted into a carrier-slide (or carrier-plate) 111 comprising a compartment connected to a reservoir 112 below the lower surface of the plate 110 by which suction may be carried out. An ‘O’-ring 113 provides fluid-tightness for the connection between a tip 117 and the plate 110. An ‘O’-ring 114 provides fluid-tightness for the connection between the plate 110 and the carrier slide 111. The fluid-tightness provided by the ‘O’-rings 113 and 114 enables suction of the content of the compartments.

During the suction by the reservoir 112, certain particular cells of the liquid present in the compartment 105, of greatest diameter, are retained by the filter 115 whereas most of the liquid and of the cells of small size are sucked out of the compartment 105, through the filter 115.

At the end of the first phase of the method for collecting cellular material, the compartments 105 are removed from the part of the device illustrated in FIGS. 1 and 2 and the tips 117 are removed from the lower openings of the compartments 105. Next, a bar-plug 120 is inserted at the tip of the compartments 105 of syringe form. This is a bar with four perforations which receive the lower tip of the compartments. A plastic film is bonded to the lower face of this bar-plug 120, and constitutes a fluid-tight membrane, preferentially adhesive.

The content of the compartment 105 is thus kept away from the environment until the collection of the cellular material of the particular cells.

At the second phase of the method, the bar 116, bearing the compartments 105, which are associated with the bar of plugs 120, is made to press on a rack 133 of plastics material. Next, the lysis of the cells present in the compartment 105 is carried out in an oven. To that end, after the addition of the reagents for the lysis of the cell membranes of the cells searched for, the rack 133, holding the compartments 105 vertically, is transported into an oven. On coming out of the oven, after cooling, the tubes of Eppendorf type are positioned on the lower support 131 of the gantry 130. The bar 116 bearing the compartments 105, associated with the bar of plugs 120, is positioned on the gantry 130 and the gantry 130 is placed on the lower support 131.

Next by the upper opening 106 of each compartment 105, there is inserted a plunger 140 provided with a central axial awl 141 ending in a point 142. Preferentially, in cross-section that point 142 is star-shaped, for example with four arms, the point then being cruciform.

Below each compartment 105 an Eppendorf type tube 125 is positioned, held in position by a shelf of the gantry 130.

FIGS. 3 and 4 illustrate the successive respective positions of the plunger 140 and of the awl 141 during the second phase of the method. At the left of each of these FIGS. 3 and 4, the plunger 140 and the awl 141 are practically entirely out of the compartment 106 and the upper part of the awl 141 projects vertically above the plunger 140.

Next, by application of a rotation, the awl is passed from a security position in which the punch 141 cannot slide longitudinally within a plunger 140 on account of a mechanical abutment, to an activation position in which the punch can slide longitudinally within the plunger 140. Next, a vertical force is applied downwardly on the apex of the awl 141, and the point 142 of the awl 141 is made to move down into the body of the compartment 106, towards the filter 115. By continuing this downward movement, the point 142 first of all pierces the filter 116 then the membrane 120 until it slightly enters the tube 125. The awl 141 is next held in position by contact on the lower opening of the compartment 105. Finally, as represented on the right of FIGS. 3 and 4, while keeping the awl 141 in place, a downwards force is applied to the plunger140 in order for it to push the content of the compartment 105 towards the tube 125, through the hole formed in the filter 115 and in the membrane 120 by the point 142 of the awl.

The Eppendorf type tubes 125 are next removed from the lower support 131, by withdrawal, towards the top of the gantry 130, of the compartments 105 and of the membrane 120, for analysis of the genetic material in particular the DNA or RNA of the cells of interest, collected in those tubes 125, in a way that is known per se.

The plate 110, the rack 133, the gantry 130 and the support 131 are re-usable.

In the second embodiment, illustrated with regard to FIGS. 1, 2, 6 and 7, a support 131 of the gantry 130 is replaced by a support 132 having eight openings instead of four. The four Eppendorf type tubes 125 are replaced by a bar of eight Eppendorf type tubes 126 of smaller capacity, typically 0.25 ml. Instead of 1.5 ml. These Eppendorf type tubes are adapted for another way of collecting cellular material making it possible to extract the genetic material directly on the filter but in a smaller volume. Such a volume may then be contained in Eppendorf type tubes directly adapted for an apparatus allowing real time RT-PCR (acronym for “reverse transcription polymerase chain reaction”). It should be noted that, when using eight Eppendorf type tubes, four tubes serve to collect genetic material and four tubes serve as positive or negative controls.

As can be seen in FIG. 9, the method of the present invention first of all comprises, in known manner, a step 200 of taking a sample of liquid to analyze, for example blood to which dilution or filtration may possibly be applied.

During a step 205, a fixation buffer without formaldehyde is applied, in order specifically to fix, that is to say harden, the particular cells searched for, without altering their genetic material.

For example, the fixation buffer is composed of “PBS”, a buffer of phosphates of saponin for lysing the red blood cells, of BSA, bovine serum albumin for preserving the morphology of the cells, of ETDA chelating agent, of calcium, of NaOH for adjusting the pH to 7.2, and of RCL2, a cell fixative not altering their genetic material. It should be noted that formaldehyde is not used since it introduces breakages in the genetic material.

During a step 210, the liquid resulting from step 205 is placed in a compartment 105 which ends with a filter with micropores of a diameter intermediate between that of the cells searched for and that of the other cells of the liquid sample. As a variant, the fixation of step 205 is carried out within the compartment 105, after step 210.

During a step 215, suction is applied by reduced pressure below the filter. Most of the liquid as well as the cells of diameter less than that of the pores of the filter 115 then pass through the filter 115. On the other hand, the cells searched for of diameter less than that of the pores of the filter 115 are retained above the filter 115, in the compartment 105.

During a step 220, the compartments are removed from the plate 110, the tips 117 are removed from the compartments 105 and the content remaining in the compartment 105 is isolated by plugging the lower opening with a bar-plug that is covered, in its lower portion, with an adhesive membrane 120.

During a step 225, the compartments 105, associated both with the bar of plugs 120 and with the bar 116, are inserted into the rack 133, the bar 116 being held by that rack 133.

During a step 230, lysis of the cells retained on the filter is carried out, according known techniques. To that end, after the addition of the reagents for the lysis of the cell membranes of the cells searched for, the rack 133, holding the compartments 105 vertically, is kept in an oven for a known time.

After removal from the oven, during a step 232, the rack 133, the bar 116, the compartments 105 and the bar of plugs 120 are removed and placed on the gantry 130.

During a step 235, each compartment 105 is placed above an Eppendorf type tube 125, or 126, by positioning that tube on the lower support, 131 or 132 respectively, then the gantry 130 with the bar 116, the compartments 105 and the bar of plugs 120 is positioned on the lower part 131, or 132, respectively.

During a step 240, through the upper opening 106 of each compartment 105, there is inserted a plunger 140 provided with a central axial awl 141 moveable relative to the plunger 140 and ending, within compartment 105, by a point 142. Preferentially, in transverse cross-section, that point 142 is star-shaped, for example with four arms, the point then being cruciform.

During a step 245, the awl is turned to take it out of its security position. Next, the upper part of the awl 141 is pushed to make it move down along the longitudinal axis of the compartment 105, while being guided by the plunger 140. During this longitudinal movement, the point 142 of the awl 141 successively perforates the filter 115 and the plug or the adhesive membrane 120.

During a step 250, the rest of the plunger 140 is pressed in order for the remaining content of the compartment 105 to pass through the filter 115 and the plug or the membrane 120, and reach the Eppendorf type tube, by the opening surrounding the point 142 of the awl 141.

Thus, the genetic material of the particular cells is extracted without deterioration via the filter perforation.

During a step 255, the support, 131 or 132, and the Eppendorf type tubes, 125 or 126, are removed after withdrawal, towards the top of the gantry 130, of the compartments 105 and of the membrane 120.

During a step 265, an analysis of the genetic material, in particular the DNA and the RNA of the cells searched for, collected in those tubes 125 or 126, in a manner known per se.

As may be understood on the reading of the description, the method and the device of the present invention make it possible to collect, in conditions compatible with routine laboratory examinations, a large proportion of the genetic material of the cells considered, in good state, even if the sample only comprises a single cell searched for.

1. A method for collecting cellular material of particular cells present in a liquid, characterized in that it comprises:
a step (210) of introducing the liquid into a compartment (105) via an upper opening (106) of the compartment, said compartment having a lower opening (107), a filter (115) being positioned between the two openings which has micropores of a diameter intermediate between that of said particular cells and that of other cells,
a filtering step (215) during which most of the liquid and of said other cells passes through the filter,
a step (230) of lysing the cells retained on the filter and
a step (245, 250) of collecting cellular material from the lysed cells, on the filter.

2. A method according to claim 1, characterized in that it comprises, prior to said filtering step (215), a step (205) of applying a fixation buffer without formaldehyde to the liquid containing the particular cells.

3. A method according to claim 1, characterized in that, during the introducing step (210), the compartment (105) has the general form of a syringe in which the filter (115) is positioned between the two openings.

4. A method according to any claim 1, characterized in that, during the filtration step (215), suction is applied by pressure reduction below the filter (115).

5. A method according to claim 1, characterized in that, following the filtering step (215) and prior to the collecting step (245, 250), the compartment is placed on an Eppendorf type tube (125, 126).

6. A method according to claim 1, characterized in that, prior to the collecting step (245, 250), a plunger is positioned in the upper opening (106) of the compartment (105), comprising a central awl (140) moveable within the plunger.

7. A method according to claim 6, characterized in that, the moveable central awl (141) has a pointed lower end (142).

8. A method according to claim 7, characterized in that, the pointed lower end (142) of the central awl (141) is star-shaped.

9. A method according to claim 6, characterized in that, during the collecting step (245, 250), the upper part of the awl (141) is pressed vertically downwards to perforate the filter with the point (142) of the awl.

10. A method according to claim 6, characterized in that, the plunger (140) comprising a means for longitudinal blocking of the awl (141), during the collecting step (245, 250), the awl is made to pivot within the plunger to free said blocking means, before pressing vertically on the upper part thereof.

11. A method according to claim 6, characterized in that, during the collecting step (245, 250), the plunger (141) is pressed to pass the remaining content of the compartment (105) into a tube (125, 126) placed below the compartment.

12. A method according to claim 1, characterized in that, following the filtering step (215) and prior to the collecting step (245, 250), the content of the compartment (105) is isolated by plugging the lower opening (107) with a membrane.

13. A method according to claim 12, characterized in that said membrane is positioned below a bar (120) surrounding the lower opening (107) of each compartment (105).

14. A method according to claim 12, characterized in that said membrane is an adhesive membrane.

15. A method according to claim 6, characterized in that, during the collecting step (245, 250), the upper part of the awl (141) is pressed to perforate the membrane isolating the content of the compartment (105).

16. A method according to claim 1, characterized in that the filter (115) is made of polycarbonate treated with a hydrophilic surface treatment.

17. A method according to claim, 1, characterized in that the filter (115) has a 7.5 μm baseline pore diameter.

18. A device for collecting genetic material of particular cells present in a liquid, characterized in that it comprises:
a compartment (105) comprising an upper opening (106) and a lower opening (107), a filter (115) being positioned between the two openings which has micropores of a diameter intermediate between that of said particular cells and that of other cells,
a means for introducing the liquid into said compartment via the upper opening,
a filtering means (111, 112) for passing most of the liquid and of said other cells through the filter,
a means for lysing the cells retained on the filter and
a means for collecting cellular material from the lysed cells, on the filter.
